(19) United States
(12) Patent Application Publication
(10) Pub. No.: US 2002/0094958 A1
(43) Pub. Date: Jul. 18, 2002

(54) Materials for lysosome modulation and methods of use thereof

(76) Inventor: Ben A. Bahr, Stafford Springs, CT (US)

Correspondence Address: ALIX YALE & RISTAS LLP, 750 MAIN STREET, SUITE 600, HARTFORD, CT, 06103

(21) Appl. No.: 10/056,666

(22) Filed: Oct. 29, 2001

Related U.S. Application Data
(60) Provisional application No. 60/244,327, filed on Oct. 30, 2000.
(60) Provisional application No. 60/254,778, filed on Dec. 11, 2000.

(51) Int. Cl. A61K 38/06; A61K 38/05; A61K 31/655; A61K 31/397; A61K 31/445; A61K 31/401

(52) U.S. Cl. 514/18; 514/19; 514/150; 514/210.17; 514/330; 514/423

(57) ABSTRACT

Disclosed are compounds and methods of use thereof for modulating lysosome function. Also disclosed is use of the compounds to treat neurodegenerative events and to study lysosomal function.

### FIELD OF THE INVENTION

This invention relates generally to lysosomal processes. More particularly, the present invention relates to methods and compounds for studying and modulating lysosomal function.

### BACKGROUND OF THE INVENTION

Many types of neurodegeneration are accompanied by aberrant protein processing events and a loss of communication between neurons at their connections called synapses. Abnormal protein processing, in fact, has been linked to the disruption of brain synapses. Lysosomes, organelles that contain hydrolytic enzymes, are the neurons' own garbage disposals responsible for degrading and recycling old and damaged proteins throughout the life of the cell. Accordingly, disruption of lysosomal function causes the intracellular buildup of both protein fragments and aggregates. Many studies have indicated that lysosomal disturbances facilitate the production of abnormal material including the amyloid species and neurofibrillary tangles found in Alzheimer's patients. Parkinson's disease, the second most common neurodegenerative disorder, and several lysosomal storage diseases that cause mental retardation, also exhibit the buildup of aggregated material inside neurons. Furthermore, lysosomes have been identified as the site at which aberrant material is produced in neurons, and lysosomal disturbances have been shown to induce many types of neurodegenerative processes including pathophysiology, axonal and dendritic transport failure, and concomitant synaptic deterioration. Lysosomes also are implicated in the synapse loss evident during brain aging; that is, as brain neurons age, lysosomal processing becomes less efficient and neurons become increasingly vulnerable to neurodegenerative events.

### SUMMARY OF THE INVENTION

An object of the invention is to provide a method to modulate lysosomal function.

Another object of the invention is to provide compounds that can be used to modulate lysosomal function.

Yet another object of the invention is to provide pharmacological compounds and methods for their use to modulate lysosome function.

Still another object of the invention is to provide a compound and method of use to improve deteriorated synapses and their functional responses.

A further object of the invention is to provide a method to study lysosomal function.

Other objects and advantages of the invention will become apparent from the specification.

It is believed that a specific pathogenic cascade is initiated by gradual lysosomal perturbation. Such lysosomal dysfunction is followed by amyloidogenesis and modification of tubulin chemistries; hyperphosphorylation and aggregation of microtubule-associated proteins and related protein fragments such as the tau species; concomitant destabilization of microtubules; disruption of axonal and dendritic transport processes; reduction in presynaptic composition, signaling, and, thus, synapse maintenance; and corresponding deterioration of postsynaptic structures and functional responses. This cascade is believed to lead to the types of synaptic deterioration linked to cognitive decline and dementia. Indeed, many of the pathogenic steps identified above are evident in tissue samples from Alzheimer's patients, especially the abnormal processing and accumulation of proteins and protein fragments. This scenario establishes a strong correlation between lysosomal dysfunction and the gradual destruction of synaptic connections.

One aspect of the invention is a lysosome modulating compound that modulates lysosomal activity without necessarily increasing cellular content of lysosomes and/or lysosomal enzymes. Some novel lysosome modulating compounds comprise M-aaₙ—CH═N═N; M-aaₙ—CH₂—O—CO—[2-R-4-R-6-R-Phenyl] (wherein each R is independently selected); M-aaₙ—NH—CH₂—CH═N—NH—CO—NH₂; M—N═N—CO—CH₂—aaₙ—O—R; or combinations thereof; wherein:

M comprises H, benzyloxycarbonyl (“Z”), succinyl, methyloxysuccinyl, and butyloxycarbonyl;

comprises a blocked or unblocked amino acid with the L configuration, D configuration, or no chirality at the alpha-carbon, the amino acid comprising alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutyric acid, epsilon-aminocaproic acid, citrulline, hydroxyproline, homoarginine, ornithine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂—CHEt₂)—COOH, alpha-aminoheptanoic acid, NH₂—CH(CH₂—1-napthyl)—COOH, NH₂—CH(CH₂—2-napthyl)—COOH, NH₂—CH(CH₂-cyclohexyl)—COOH, NH₂—CH(CH₂-cyclopentyl)—COOH, NH₂—CH(CH₂-cyclobutyl) —COOH, NH₂—CH(CH₂-cyclopropyl)—COOH, trifluoroleucine, hexafluoroleucine, phenylalanine with its phenyl mono-, di-, or trisubstituted with K, alanine with its methyl side chain replaced with a lower alkyl side chain, alanine with its methyl side chain replaced with a lower alkyl group with an attached phenyl group, alanine with its methyl side chain replaced with a lower alkyl group with two attached phenyl groups, alanine with its methyl side chain replaced with a lower alkyl group with an attached phenyl group substituted with K, and alanine with its methyl side chain replaced with a lower alkyl group with two attached phenyl groups and at least one phenyl group substituted with K;

n comprises an integer from 1 to about 20;

R comprises H, a lower alkyl group, a lower fluoroalkyl group, benzyl, a lower alkyl group substituted with J, a lower fluoroalkyl group substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, a lower alkyl group with an attached phenyl group, a lower alkyl group with two attached phenyl groups, a lower alkyl group with an attached phenyl group substituted with K, or a lower alkyl group with two attached phenyl groups and at least one phenyl group substituted with K;

J comprises halogen, COOH, OH, CN, NO₂, NH₂, lower alkyl—OH, lower alkoxy, lower alkylamine, di-lower alkylamine, lower alkoxy—CO—, lower alkyl—O—CO—NH, and lower alkyl—S—;

K comprises halogen, lower alkyl, lower alkyl—OH, lower perfluoroalkyl, lower alkoxy, NO₂, CN, OH, CO—OH, amino, lower alkylamine, C2-12 dialkylamine, lower acyl—O—CO—NH, lower alkoxy—CO—, and lower alkyl—S—.

Unless otherwise specifically defined, the term “lysosome modulating compound” includes pharmacologically acceptable salts of the compound.

Unless otherwise specifically defined, “lower alkyl” refers to a straight, branched chain or cyclic alkyl group having from 1 to about 10 carbon atoms including, for example, methyl, ethyl, propyl, butyl, hexyi, octyl, isopropyl, isobutyl, tert-butyl, cyclopropyl, cyclohexyl, cyclooctyl, vinyl and allyl. The lower alkyl group can be saturated or unsaturated and substituted or unsubstituted. Unless otherwise specifically defined, “lower-alcohol” refers to the general formula lower alkyl-OH. Unless otherwise specifically defined, “lower-alkoxy” refers to the general formula —O-lower alkyl. Unless otherwise specifically defined, “lower alkylmercapto” refers to the general formula-S-lower alkyl. Unless otherwise specifically defined, “lower alkylamine” refers to the general formula-(NH)-lower alkyl. Unless otherwise specifically defined, “di-lower-alkylamine” refers to the general formula-N-(lower-alkyl)₂.

Substituent groups for the above moieties useful in the invention are those groups that do not significantly diminish the biological activity of the inventive compound. Substituent groups that do not significantly diminish the biological activity of the inventive compound include, for example, —OH, —NH₂, lower alkoxy, halogen, —CN, —NCS, azido, —CONH, —NHCO, sulfonamide, lower alcohol.

In one embodiment of the invention the novel lysosome modulating compound comprises benzyloxycarbonyl-Phe-ala-diazomethylketone, benzyloxycarbonyl-Phe-Phe-diazomethyl ketone, benzyloxycarbonyl-Phe-Lys-2,4,6-trimethylbenzoyloxymethylketone, benzyloxycarbonyl-Lys-diazomethylketone, H-Gly-Phe-Gly-aldehyde semicarbazone, diazoacetyl-DL-2-aminohexanoic acid-methyl ester, and combinations thereof. One preferred lysosome modulating compound comprises benzyloxycarbonyl-Phe-Ala-diazomethylketone.

In one embodiment of the invention the novel lysosome modulating compound functions as a selective antagonist for at least one cathepsin enzyme.

Another aspect of the invention is use of the above described lysosome modulating compounds to increase the cellular content of lysosomal enzymes.

The novel lysosome modulating compounds, singly or in combination, can modulate lysosomal function in a subject. Thus, a further aspect of the invention is a method of modulating lysosomal function in a subject comprising administering to the subject a therapeutically effective amount of at least one of the above described lysosome modulating compounds.

The novel lysosome modulating compounds can function to enhance cellular production of lysosomal enzymes and thereby promote degradative processing of aberrant protein fragments and aggregates. The digestive processing can be useful as a prophylaxis to prevent or reduce the risk of neurodegenerative events. Thus, still another aspect of the invention is a method of reducing the risk of a neurodegenerative disorder in a subject comprising administering to the subject a therapeutically effective amount of at least one of the above described lysosome modulating compounds.

The novel lysosome modulating compounds can function to enhance cellular production of lysosomal enzymes and thereby promote degradative processing of aberrant protein fragments and aggregates. The digestive processing can be useful in the treatment of neurodegenerative events. Thus, still another aspect of the invention is a method of treatment of a neurodegenerative disorder in a subject comprising administering to the subject a therapeutically effective amount of at least one of the above described lysosome modulating compounds.

The novel lysosome modulating compounds can modulate lysosomal function in a tissue culture. Thus, yet another aspect of the invention is a method of studying lysosomal function in a tissue culture comprising administering to the tissue culture an amount of at least one of the above described lysosome modulating compounds.

### BRIEF DESCRIPTION OF THE DRAWINGS

Other objects and advantages of the invention will be evident to one of ordinary skill in the art from the following description made with reference to the accompanying drawings, in which:

FIG. 1A is a graph showing a progressive decline in the cellular concentration of pathogenic protein species upon administration of benzyloxycarbonyl-Phe-Ala-diazomethylketone (PADK) to brain slice cultures, made from the hippocampal region, over a 20-day period;

FIG. 1B is a graph showing progressive increase in the cellular concentration of non-pathogenic protein species upon administration of the Phe-Ala-diazomethylketone derivative to hippocampal slice cultures over a 20-day period;

FIG. 1C is a graph showing stable levels of the synaptic marker GluR1 and a progressive increase in the cellular concentration of the lysosomal enzyme cathepsin D upon administration of PADK to hippocampal slice cultures over a 20-day period;

FIG. 2 is a graph showing synaptic marker GluR1 concentrations upon administration of the general lysosomal disruptor chloroquine to hippocampal slice cultures over a 6-day period (solid line defined by open circles). During the 2-day period after removal of chloroquine, GluR1 concentrations continue to decline in untreated slices (dotted line defined by open triangles), while significant and progressive recovery of GluR1 is observed upon administration of the lysosomal modulator PADK (dotted line defined by closed triangles);

FIG. 3A is a graph showing significant recovery of the postsynaptic marker (GluR1) after administration of the lysosomal modulator PADK to hippocampal slice cultures previously treated with the lysosomal disruptor chloroquine (first bar), but no such recovery after administration of Ampakine (second bar) or memantine (third bar);

FIG. 3B is a graph showing significant recovery of the presynaptic marker synaptophysin after administration of PADK to hippocampal slice cultures previously treated with chloroquine (first bar), but insignificant or no recovery after administration of Ampakine (second bar) or memantine (third bar);

FIG. 4 is a picture of immunoblots showing upmodulation of the lysosomal enzyme cathepsin D (designated CD in the figure) by benzyloxycarbonyl-Phe-Ala-diazomethylketone (PADK), and corresponding reduction in neuropathogenic tau species of 55-69 kDa induced in the in vitro method of lysosomal dysfunction mediated by chloroquine (CQN);

FIG. 5 is a picture of immunoblots showing upmodulation of the lysosomal enzymes cathepsin B (designated CB), cathepsin D (CD), and cathepsin S (designated CS) by PADK. Similar upmodulation has been observed with regards to the lysosomal enzyme beta-glucuronidase;

FIG. 6 is a graph where neuronal ability to transport an intracellular protein through dendrites was scored in cultured tissue slices by two independent observers. The slice groups consisted of nontreated control slices (top panel), slices subjected to chloroquine-mediated lysosomal dysfunction for 6 days followed by 2 days of media alone (middle panel), and slices subjected to the lysosomal dysfunction followed by 2 days of PADK treatment as in FIG. 2 (bottom panel);

FIG. 7 summarizes the pathogenic changes that stem from chloroquine-induced lysosomal dysfunction and the compensatory responses facilitated by treatment with the lysosomal modulator benzyloxycarbonyl-Phe-Ala-diazomethylketone; and

FIG. 8 is a graph showing upmodulation of the lysosomal enzyme cathepsin D in vivo. Cathepsin D concentrations in brain tissue samples from a group of 10-11 untreated rats were compared to those determined in a group of 5 rats treated orally with benzyloxycarbonyl-Phe-Ala-diazomethylketone over a 7-day period.

### DETAILED DESCRIPTION OF THE INVENTION

Disrupting lysosomal function in cultured brain slices induces degenerative events similar to those found in Alzheimer's disease, including intracellular protein accumulation and blockage of transport systems needed to maintain neuronal health by delivering vital materials to synaptic connections. During the lysosomal disturbance, the brain slices also express a small increase in the levels of digestive enzymes used by lysosomes to process proteins. Interestingly, similar small increases in lysosomal enzymes have been reported to occur in the brains of Alzheimer's patients. Such lysosomal enzyme proliferation found in Alzheimer's disease likely represents an internal repair system that is activated in response to the pathology. While this type of enzymatic response should enhance the ability of lysosomes to digest components of protein deposits, the degree of the protein deposition in vivo or in the brain slices precludes any effects of such a compensatory response. Pharmacological modulation of lysosomal function, however, has been discovered to reverse protein accumulation and synaptic deterioration. Lysosomal modulation, thus, provides a treatment for neurodegenerative events including those underlying Alzheimer's disease, Parkinson's disease, and lysosomal storage disorders.

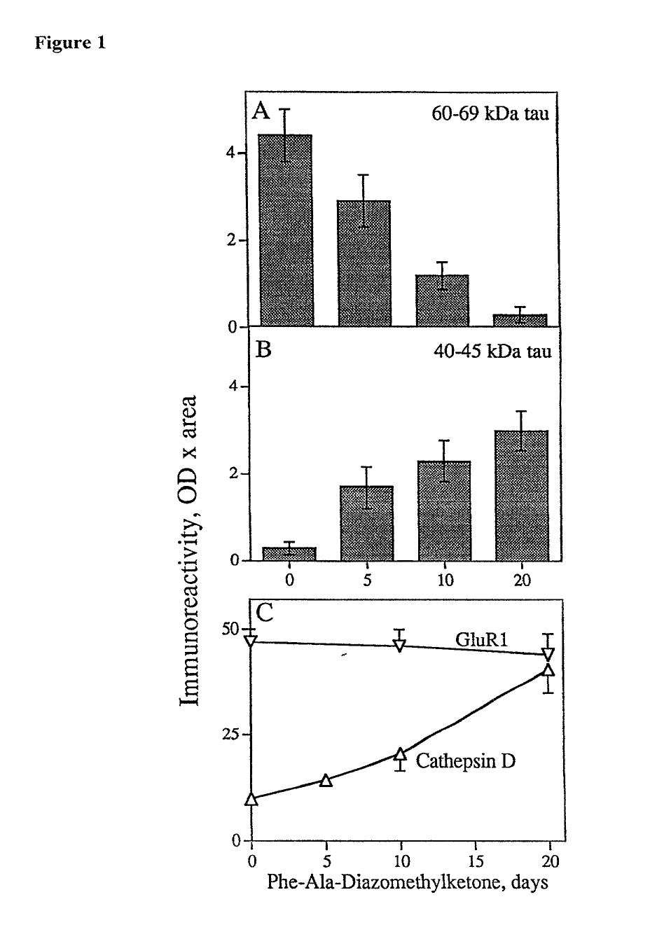

One aspect of the invention is the use of compounds to modulate lysosomal activity. The modulation of lysosomal activity may be, but is not necessarily, accompanied by an increase in cellular content of lysosomes or lysosomal enzymes. Some inventive lysosomal modulating compounds comprise the previously described formulas (1) M-aaₙ—CH═N═N, (2) M-aaₙ—CH₂—O—CO—[2-R-4-R-6-R-Phenyl], (3) M-aaₙ—NH—CH₂—CH═N—NH—CO—NH₂, (4) M-N ═N—CO—CH₂—aaₙ—O—R, and combinations thereof. An example of formula (1) is benzyloxycarbonyl-Phe-Ala-diazomethylketone (PADK). As discussed in more detail later, PADK was applied to hippocampal slice cultures at a continuous concentration of 10 μM over a 20-day period. Fresh media and PADK compound were applied every two days. Surprisingly, PADK caused a reduction in pathogenic hyperphosphorylated species of the microtubule-binding protein tau (FIG. 1A) and a corresponding increase in non-pathogenic tau isoforms (FIG. 1B). These two events indicate efficient tau turnover/processing and were evident after only 5 days of PADK treatment, a time point at which the concentration of the lysosomal enzyme cathepsin D was not significantly altered (FIG. 1C). This suggests that lysosomal modulation dissipates pathogenic precursors of neurofibrillary tangles independent of an increase in the cellular content of lysosomes or their enzymes.

Another aspect of the invention is the provision of compounds to increase cellular content (upmodulation) of lysosomal enzymes. As used herein “upmodulation” is an increase in the cellular content of lysosomal enzymes or in the content of lysosomes. Some inventive lysosomal modulating compounds are comprised by the previously described formulas (1) M-aaₙ—CH═N═N, (2) M-aaₙ—CH₂—O—CO—[2-R-4-R-6-R-Phenyl], (3) M-aaₙ—NH—CH₂—CH N—NH—CO—NH₂ and (4) M—N═N—CO—CH₂-aaₙ—O—R. Examples of compounds include, formula (1) benzyloxycarbonyl-Phe-Ala-diazomethylketone and benzyloxycarbonyl-Phe-Phe-diazomethylketone; formula (2) is benzyloxycarbonyl-Phe-Lys-2,4,6-trimethylbenzoyloxymethylketone; formula (3) is H-Gly-Phe-Gly-aldehyde semicarbazone; formula (4) is diazoacetyl-DL-2-aminohexanoic acid-methyl ester. When these compounds were individually applied to hippocampal slice cultures, each facilitated the upmodulation of lysosomal enzyme levels (Table 1). Each of the compounds is available from BACHEM Biosciences Inc. of King of Prussia, Pa. Presently, PADK is the preferred compound for modulation of lysosomal activity.

TABLE 1

| | | upmodulated |
|---|---|---|
| | | cathepsin |
| Lysosomal Modulator | | types |
| (1) | benzyloxycarbonyl-Phe-Ala-diazomethylketone | B, D, S |
| (2) | benzyloxycarbonyl-Phe-Phe-diazomethylketone | B, D |
| (3) | benzyloxycarbonyl-Phe-Lys- | D |
| | 2,4,6-trimethylbenzoyloxymethylketone | |
| (4) | H-Gly-Phe-Gly-aldehyde semicarbazone | B, D |
| (5) | diazoacetyl-DL-2-aminohexanoic acid-methyl ester | D |

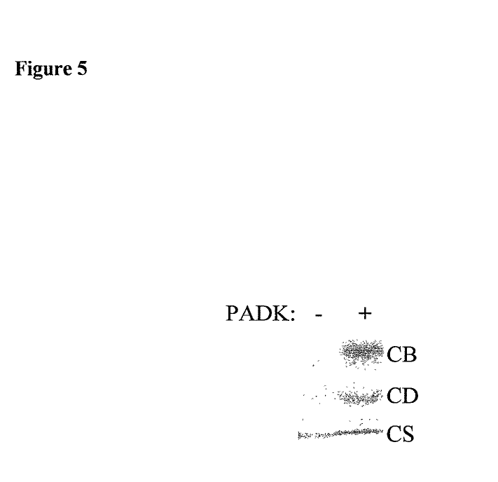

The inventive lysosomal modulating compounds can be used as selective inhibitors targeting members of the cathepsin family of lysosomal hydrolases. Surprisingly, neurons respond to mild inhibition of cathepsins by enhancing their production of a variety of lysosomal enzymes including cathepsin D (FIG. 1C and FIG. 5). Cathepsin D is the lysosomal hydrolase identified as being most involved in digesting pathogenic protein species found in Alzheimer's-affected brains.

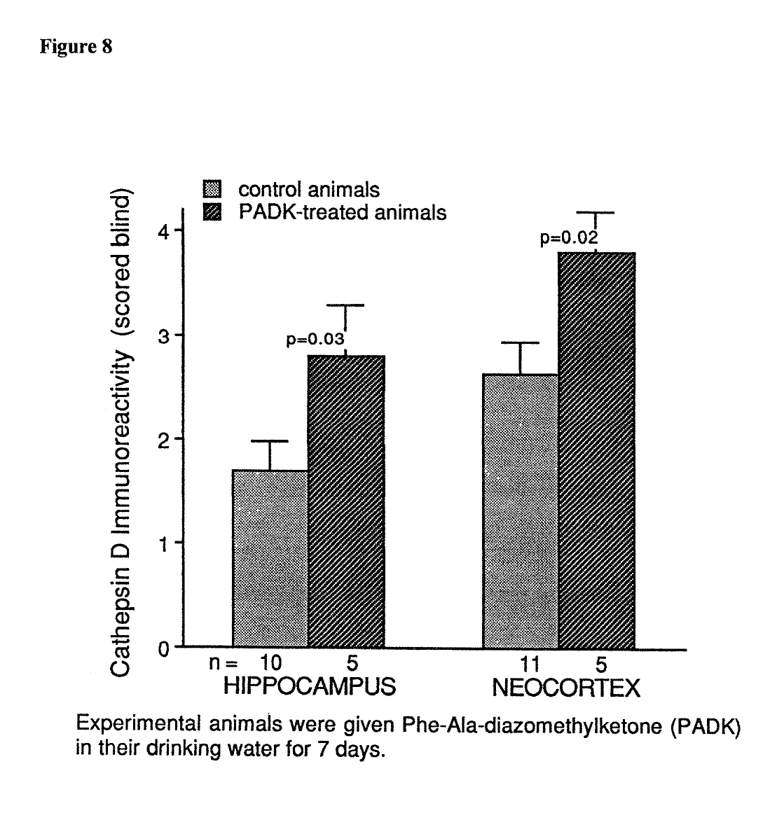

A further aspect of the invention is a method of modulating lysosomal function in a subject comprising administering to the subject a therapeutically effective amount of at least one inventive lysosome modulating compound or a physiologically acceptable salt thereof. As discussed in more detail later, the lysosome modulating compound benzyloxycarbonyl-Phe-Ala-diazomethylketone (PADK) was added to the drinking water of a first group of 5 adult male rats in an amount corresponding to 3 to 4 mg PADK/kg rat weight/day. A second group of 10-11 adult male rats was treated similarly with the exception that the drinking water did not contain PADK. After seven days, hippocampal and neocortical tissue was isolated from the first and the second groups. The concentration of the lysosomal enzyme cathepsin D in tissue samples was assessed by immunoblot for each of the groups. As shown in FIG. 8, animals from the group treated with PADK showed significant increases in lysosomal enzyme concentration as compared to animals from the untreated group.

As used herein, a “therapeutically effective amount” of a compound is the quantity of a compound which, when administered to a subject, results in a sufficiently high level of that compound in the subject to cause a discernible therapeutic effect. Typically, a “therapeutically effective amount” of an inventive compound is believed to range from about 0.5 mg/kg/day to about 100 mg/kg/day. Some therapeutically effective amounts for the inventive compounds are discussed below.

As used herein, a “subject” refers to an individual or an animal. An “individual” refers to a human. An “animal” refers to, for example, veterinary animals, such as dogs, cats, horses and the like, and farm animals, such as cows, pigs and the like.

The compound of the present invention can be administered by a variety of known methods, including orally, rectally, or by parenteral routes (e.g., intramuscular, intravenous, subcutaneous, nasal or topical). The route of administration will determine the form in which the compounds are administered. Such forms include, but are not limited to, capsular and tablet formulations (for oral and rectal administration), liquid formulations (for oral, intravenous, intramuscular, subcutaneous, ocular, intranasal, inhalation based and transdermal administration) and slow releasing microcarriers (for rectal, intramuscular or intravenous administration). The formulations can also contain a physiologically acceptable vehicle and optional adjuvants, flavorings, colorants and preservatives. Suitable physiologically acceptable vehicles may include, for example, saline, sterile water, Ringer's solution., and isotonic sodium chloride solutions. The specific dosage level of active ingredient will depend upon a number of factors, including, for example, biological activity of the particular preparation, age, body weight, sex and general health of the individual being treated.

Still another aspect of the invention is a method of treatment of a neurodegenerative disorder in a subject comprising administering to the subject a therapeutically effective amount of at least one lysosome modulating compound. The inventive method provides a pronounced increase in lysosomal capacity, to prevent abnormal protein processing, and to attenuate synaptic degeneration and dysfunction. The upmodulation of lysosomal enzymes will help offset the accumulation of aberrant proteins found associated with various pathologies and, thus, provide a treatment to delay or slow neurodegenerative events.

Lysosomal modulating compounds were identified that unexpectedly stimulate lysosomal activity and/or cellular feedback processes for the upregulation of lysosomal hydrolase levels. These modulating compounds were applied to cultured rat brain slices at concentrations as low as 3 μM and were found to cause marked upmodulation of a variety of lysosomal enzymes including cathepsins (FIG. 1C and FIG. 5). Surprisingly and importantly, as the lysosomal enzyme was modulated in the slice cultures by the inventive compounds over the extended period, pathogenic hyperphosphorylated species of the microtubule-binding protein tau were gradually reduced in concentration as shown in FIG. 1A. This suggests that lysosomal modulation dissipates pathogenic precursors of neurofibrillary tangles and thereby reduces the risk of abnormal protein accumulation within neurons. Further surprisingly, a gradual increase in non-pathogenic tau isoforms also corresponded with the changes in lysosomal enzyme and hyperphosphorylated tau levels as shown in FIG. 1B. Thus the inventive lysosomal modulation promotes efficient tau turnover/processing and thereby reduces the ability of abnormally phosphorylated tau to drive non-pathogenic species off of microtubules and into the aggregated state. The non-pathogenic tau molecules would then be more readily available to stabilize microtubules and their transport mechanisms that are important for neuronal functions.

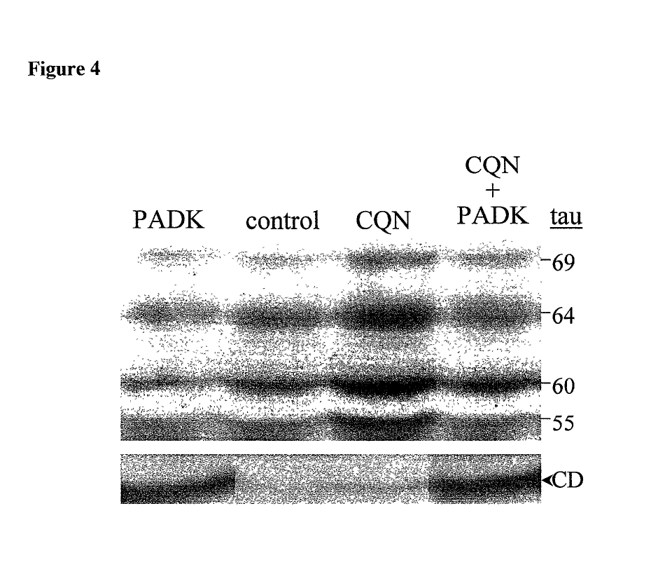

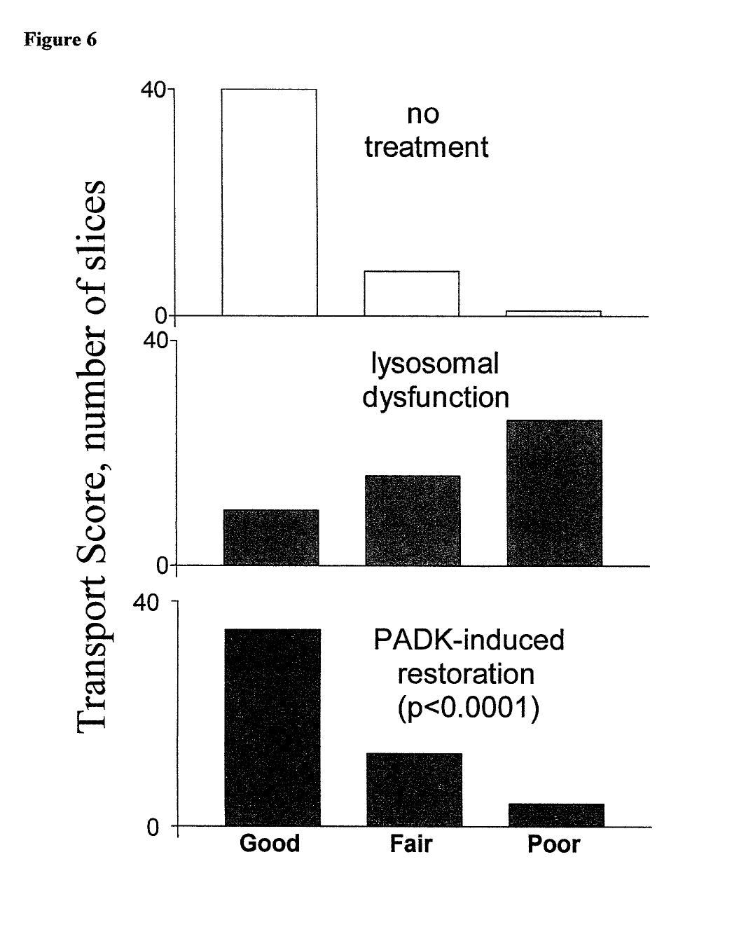

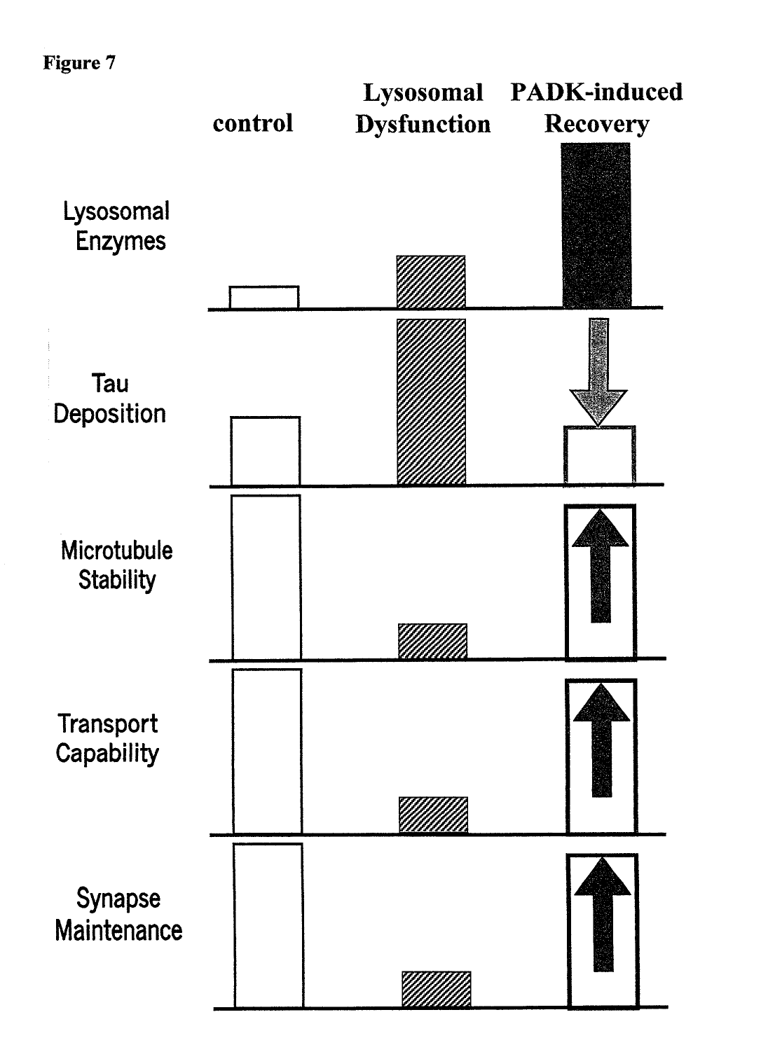

To test for protection against abnormal protein processing, lysosomal function was perturbed with the lysosomal disruptor chloroquine in cultured brain slices, after which recovery was assessed in the presence or absence of the lysosomal modulator benzyloxycarbonyl-Phe-Ala-diazomethylketone (PADK). As shown in FIG. 1C, benzyloxycarbonyl-Phe-Ala-diazomethylketone applied alone to cultures does not negatively influence synaptic maintenance over extended additions of the compound. Under conditions of lysosomal dysfunction and associated synaptic pathology, however, the lysosomal modulation produced increased lysosomal capacity to process aberrant and pathogenic protein species (FIG. 1A, FIG. 4, and FIG. 7). Without the lysosomal modulator benzyloxycarbonyl-Phe-Ala-diazomethylketone, protein transport systems needed for neuronal health remained impaired in the brain slices, whereas little impairment was evident among the slices that received the modulator (FIG. 6).

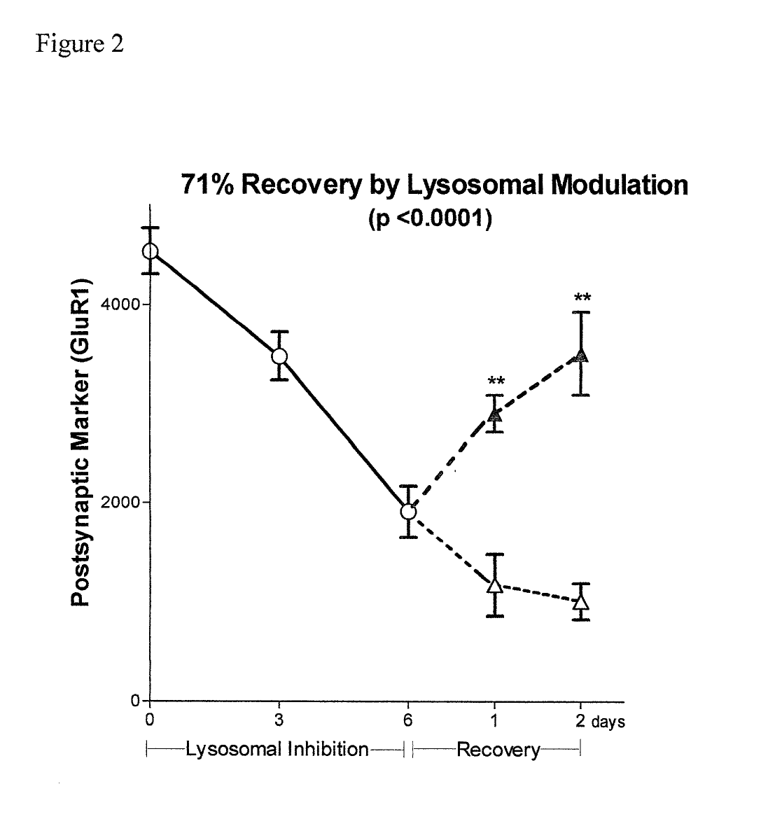

Upon removal of the lysosomal disrupter chloroquine from the periodic feeding media, the slice cultures exhibit slow or no synaptic recovery (see the dashed line of FIG. 2 defined by the open triangles). Lysosomal dysfunction caused by the chloroquine addition creates aberrant protein processing/aggregation within neurons, followed by disruption of cellular transport mechanisms needed for replenishing synapses with new proteins and organelles for functional maintenance. As shown by FIG. 2, even after removal of the lysosomal disrupter, there was too much aberrant material accumulated in neurons for their lysosomes to process in order to reestablish important transport capability needed for synaptic recovery.

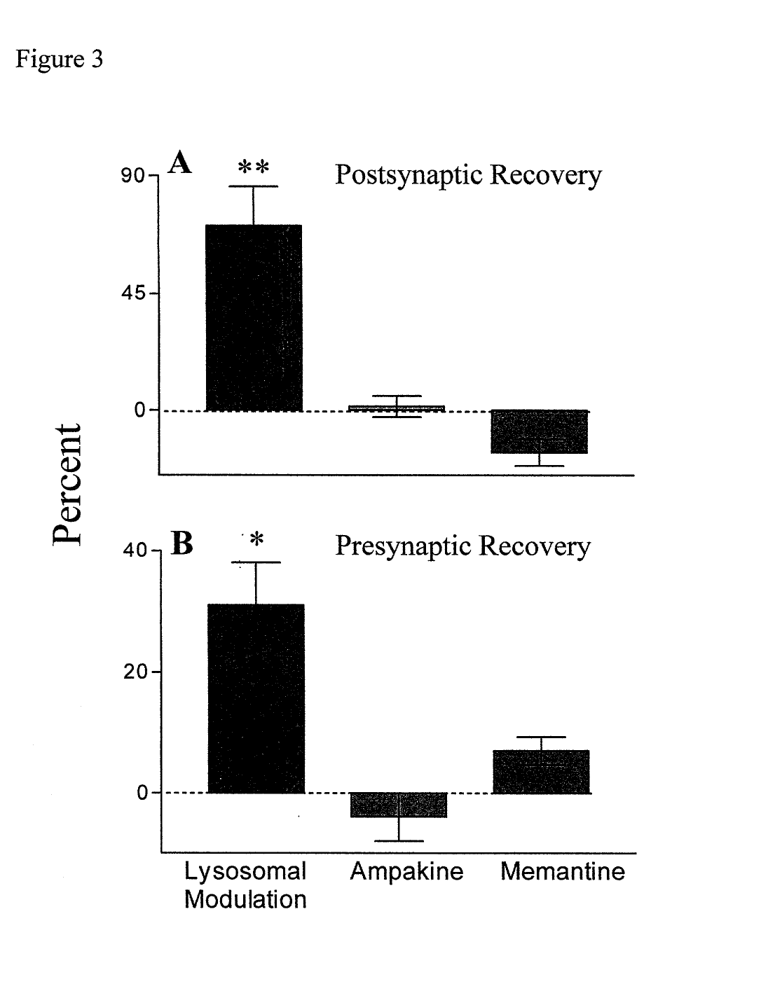

In a second group of slice cultures, after removal of the lysosomal disruptor chloroquine the lysosomal modulator benzyloxycarbonyl-Phe-Ala-diazomethylketone is added in order to increase lysosome activity. The increased lysosome activity and/or enhanced lysosomal enzyme levels function to process accumulated aberrant material, thereby leading to an increase in synaptic maintenance as indicated by recovery of the neurotransmitter receptor subunit concentration (see the dashed line of FIG. 2 defined by closed triangles). The lysosomal modulation facilitated the recovery of pre- and postsynaptic markers that were diminished due to lysosomal dysfunction (FIGS. 2 and 3). This result also indicates that neuron transport systems have been reestablished to support the maintenance of synapses.

The chloroquine addition induces lysosomal dysfunction. The lysosomal dysfunction is believed to be followed by modification of tubulin chemistries; hyperphosphorylation and aggregation of pathogenic microtubule-associated proteins and protein fragments such as the tau species; concomitant destabilization of microtubules; disruption of axonal and dendritic transport processes; reduction in presynaptic components and, thus, synapse maintenance; and corresponding deterioration of postsynaptic structures and functional responses. This cascade is reversed by lysosomal modulation (FIGS. 2 and 7).

Yet another aspect of the invention is a method for studying lysosomal function in a tissue culture comprising administering to the tissue culture an amount of at least one lysosomal modulating compound. The method advantageously uses a culture of tissue slices from rat brain hippocampus since age-related synaptic changes and Alzheimer-type pathogenesis are concentrated there.

The method comprises treatment of a neural tissue culture with a lysosomal disruptor (e.g., chloroquine) to initiate lysosome dysfunction in the culture. The culture is treated with a lysosomal disruptor in order to reproduce features characteristic of neurodegeneration, especially those found in Alzheimer's disease (FIG. 7). Preferably, chloroquine is utilized for the lysosomal disruptor due to its broad targeting of pH-dependent enzymes by disrupting the proton gradient of lysosomes. Chloroquine is added to the culture for an extended period of time (the addition time period) to cause gradual cellular changes.

After a predetermined time, the lysosomal disrupter is removed from the culture and synaptic marker concentrations are determined. Subsequently, small amounts of compounds are administered to the neural tissue to modulate lysosomal enzymes. After such treatment, concentrations of synaptic markers in the neural tissue are determined, and the treated and untreated synaptic marker concentrations are compared to determine synaptic recovery (see FIG. 2). Thus by monitoring decline (during disruptor addition) and subsequent increase (during compound addition) of synaptic markers, synaptic degeneration and recovery can be assessed, and the efficacy of the added compounds can be measured.

Hippocampal slice cultures exhibit many key features of the adult brain including native circuitry, cellular organization, synaptic density, and memory-related plasticity. When subjected to a variety of insults these cultures express a pathological responsiveness, which, particularly at the synapse level, is similar to that expected from in vivo studies. Moreover, the pathogenic responses to lysosomal dysfunction are similar in sensitivity and temporal relationship to those found in the aged human brain and Alzheimer's disease.

It should be understood that the following examples are included for purposes of illustration so that the invention may be more readily understood and are in no way intended to limit the scope of the invention unless otherwise specifically indicated.

### EXAMPLE 1

Benzyloxycarbonyl-Phe-Ala-diazomethylketone (PADK) was applied to hippocampal slice cultures at a continuous concentration of 10 μM over a 20-day period. Fresh media and PADK compound were applied every two days. As shown in FIG. 1C, the application of PADK stably enhanced cathepsin D levels over the 20-day period without any reduction in synaptic marker (GluR1). Surprisingly and importantly, during the lysosomal modulation period, pathogenic hyperphosphorylated species of the microtubule-binding protein tau were gradually reduced in concentration as shown in FIG. 1A. This suggests that lysosomal modulation dissipates pathogenic precursors of neurofibrillary tangles and prevents synaptic deterioration. Further surprisingly, a gradual increase in non-pathogenic tau isoforms also corresponded with the changes in lysosomal enzyme and hyperphosphorylated tau levels as shown in FIG. 1B. Thus the inventive lysosomal modulation promotes efficient tau turnover/processing and thereby reduces the ability of abnormally phosphorylated tau to drive non-pathogenic species off of microtubules and into the aggregated state. The non-pathogenic tau molecules would then be more readily available to stabilize microtubules and their transport mechanisms that are important for neuronal functions. Such an inverse correlation between the two forms of tau was also evident in the hippocampus of aged mice, where the change vs. age relationships determined for hyperphosphorylated and non-phosphorylated tau were of equal but opposite slopes. Thus, lysosomal modulators may intervene in the progression of certain types of neuronal atrophy, as well as in the cellular processes that contribute to synaptic loss and the severity of Alzheimer-type dementia.

### EXAMPLE 2

In order to induce lysosomal dysfunction, 60 μM chloroquine in media was added separately to groups of hippocampal slice cultures. The chloroquine-containing media was changed every two days. Over a 6-day period, separate groups of slices were harvested and synaptic pathology assessed by determining the content of the postsynaptic marker GluR1. As shown in FIG. 2, GluR1 concentration decreased over the 6-day exposure period indicating synaptic degeneration had occurred. In a first set of slices the chloroquine was removed from the feeding media after the 6-day addition period and synaptic recovery was assessed after one and two days by measuring GluR1 concentration. The dotted line defined by open triangles shows further decline in the synaptic marker concentration, indicating continued synaptic degeneration. Although the rate of decline slowed between the first and second day, there was no evidence of synaptic recovery.

In a second set of slices, synaptic recovery was assessed after removal of chloroquine-containing media and a single addition of 10 μM of the lysosomal modulator benzyloxycarbonyl-Phe-Ala-diazomethyiketone (PADK) in media was applied to cultures. As shown by the closed triangles, addition of PADK functioned to increase the GluR1 marker concentration, indicating recovery from synaptic degeneration. Surprisingly, the small addition of PADK provided a significant recovery from synaptic degeneration in only one day (p<0.001). After two days, the cultures exhibited a 71% recovery of GluR1 level (p<0.0001).

### EXAMPLE 3

Lysosomal dysfunction was induced by adding 60 μM chloroquine in media to separate groups of hippocampal slice cultures. The chloroquine-containing media was changed every two days for a total of six days. After the 6-day addition period, the chloroquine was removed from the feeding media of individual sets of cultures and presynaptic marker (synaptophysin) and postsynaptic marker (GluR1) concentrations assessed to provide a baseline indicated by the horizontal dotted lines in FIGS. 3A and 3B. 10 μM PADK, 10 μM Ampakine (trademark of Cortex Pharmaceuticals; Irvine, Calif.), and 10 μM memantine (derivatives of which are a trademark of Merz Pharmaceuticals; Germany) were added separately to separate chloroquine-treated cultures to assess their effects on synaptic recovery. After a 2-day recovery period, the separate groups of cultures were harvested and concentrations of presynaptic (synaptophysin) and postsynaptic (GluR1) markers were measured and compared to the reduced levels resulting from the 6-day chloroquine treatment. An increase from the baseline indicates recovery while no difference or further reduction of marker concentrations from the baseline indicates no recovery of synaptic maintenance.

The lysosomal modulation by the single addition of PADK during the recovery period functioned to produce a 70% recovery of the postsynaptic marker GluR1 concentration (p<0.0001; FIG. 3A) and an approximate 35% recovery of the presynaptic marker synaptophysin concentration (p<0.01; FIG. 3B) as compared to baseline values obtained from cultures treated for 6 days with chloroquine alone. No statistically significant recovery of the synaptic markers was produced by the addition of Ampakine or memantine (see FIG. 3A and 3B). With regard to the small decrease in GluR1 level associated with the memantine addition (shown in FIG. 3A) and the small decrease in synaptophysin level associated with the Ampakine addition (shown in FIG. 3B), it is not known whether the decreases were due to the agent added or to continued synaptic degradation after chloroquine removal as in the untreated cultures of Example 2.

Ampakine and memantine are being developed as potential therapeutic agents for stroke and Alzheimer's disease. As can be seen from FIGS. 3A and 3B, addition of PADK functions to provide significant pre- and postsynaptic recovery while addition of Ampakine or memantine proved to be statistically ineffective.

### EXAMPLES 4 AND 5

Lysosomal dysfunction was produced in hippocampal slice cultures with chloroquine as in Example 2. After 6 days of treatment followed by 2 days with media alone, enhanced levels of potentially pathogenic tau species of 55-69 kDa were demonstrated by immunoblot staining (FIG. 4, CQN lane as compared to untreated control sample) along with a small compensatory increase in cathepsin D (CD). When the chloroquine treatment was followed by 2 days of addition of the lysosomal modulator benzyloxycarbonyl-Phe-Ala-diazomethylketone (PADK), cathepsin D exhibited robust upmodulation and a corresponding reduction in the 55-69 kDa tau species occurred (FIG. 4). The PADK effect on lysosomal capacity consisted of enhanced levels of several lysosomal enzymes including cathepsin B (CB), cathepsin D (CD), and cathepsin S (CS) (FIG. 5). The PADK addition alone caused a similar upmodulation of lysosomal enzyme without altering control levels of tau isoforms (FIG. 4).

### EXAMPLES 6 AND 7

Lysosomal dysfunction was produced in hippocampal slice cultures with chloroquine as in Example 2. Separate groups of slices were assessed for transport capability by applying horse-radish peroxidase to neurons and scoring its transport from cell bodies along dendrites by two independent observers. FIG. 6 shows that transport capacity was adversely affected by the 6-day chloroquine treatment followed by 2 days of media alone (middle panel) as compared to untreated slices (top panel). In contrast, slices treated with the lysosomal modulator benzyloxycarbonyl-Phe-Ala-diazomethylketone (PADK) for two days did not exhibit transport failure when such treatment followed the 6-day chloroquine period (bottom panel). Thus, a small addition of PADK provided significant restoration of transport capability (p<0.0001) that may explain the PADK-induced synaptic recovery described in Example 2. By monitoring intracellular tau deposition, microtubule stabilization marker (acetylated tubulin), and transport capability, it appears that changes in all of these related parameters contribute to the link between lysosomal modulation and synapse maintenance (FIG. 7).

### EXAMPLE 8

The lysosomal modulator benzyloxycarbonyl-Phe-Ala-diazomethylketone (PADK) was added to the drinking water of a first group of 5 adult male rats in an amount corresponding to approximately 3 to 4 mg PADK/ kg rat weight/day. A second group of 10-11 adult male rats was treated similarly with the exception that the drinking water did not contain PADK. After seven days, hippocampal and neocortical tissue was isolated from the first and the second groups. The concentration of cathepsin D in tissue samples was assessed by immunoblot for each of the groups. As shown in FIG. 8, animals from the group treated with PADK showed significant increases in lysosomal enzyme concentration as compared to animals from the untreated group.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A method of modulating lysosomal function in a subject, comprising:
administering to the subject a therapeutically effective amount of a lysosomal modulating compound, a physiologically acceptable salt of the lysosomal modulating compound, or a combination thereof; wherein the lysosomal modulating compound comprises M-aaₙ—CH═N═N, M-aaₙ—CH₂—O—CO—[2-R-4-R-6-R-Phenyl] (wherein each R is independently selected), M-aaₙ—NH—CH₂—CH═N—NH—CO—NH₂, or M-N═N—CO—CH₂-aaₙ—O—R, wherein;
M comprises H, benzyloxycarbonyl (“Z”), succinyl, methyloxysuccinyl, and butyloxycarbonyl;
aa comprises a blocked or unblocked amino acid with the L configuration, D configuration, or no chirality at the alpha-carbon, the amino acid selected from alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutyric acid, epsilon-aminocaproic acid, citrulline, hydroxyproline, homoarginine, ornithine, sarcosine, indoline 2-carboxylic acid, 2-azetidinecarboxylic acid, pipecolinic acid (2-piperidine carboxylic acid), O-methylserine, O-ethylserine, S-methylcysteine, S—ethylcysteine, S—benzylcysteine, NH₂—CH(CH₂—CHEt₂)—COOH, alpha-aminoheptanoic acid, NH₂—CH(CH₂—1-napthyl)—COOH, NH₂—CH(CH₂—2-napthyl)—COOH, NH₂—CH(CH₂-cyclohexyl)—COOH, NH₂—CH(CH₂-cyclopentyl)—COOH, NH₂—CH(CH₂-cyclobutyl)—COOH, NH₂—CH(CH₂-cyclopropyl)—COOH, trifluoroleucine, hexafluoroleucine, phenylalanine with its phenyl mono-, di-, or trisubstituted with K, alanine with its methyl side chain replaced with a lower alkyl side chain, alanine with its methyl side chain replaced with a lower alkyl group with an attached phenyl group, alanine with its methyl side chain replaced with a lower alkyl group with two attached phenyl groups, alanine with its methyl side chain replaced with a lower alkyl group with an attached phenyl group substituted with K, and alanine with its methyl side chain replaced with a lower alkyl group with two attached phenyl groups and at least one phenyl group substituted with K;
n comprises an integer from 1 to about 20;
R comprises H, a lower alkyl group, a lower fluoroalkyl group, benzyl, a lower alkyl group substituted with J, a lower fluoroalkyl group substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, a lower alkyl group with an attached phenyl group, a lower alkyl group with two attached phenyl groups, a lower alkyl group with an attached phenyl group substituted with K, or a lower alkyl group with two attached phenyl groups and at least one phenyl group substituted with K;
J comprises halogen, COOH, OH, CN, NO₂, NH₂, lower alkyl-OH, lower alkoxy, lower alkylamine, di-lower alkylamine, lower alkoxy—CO—, lower alkyl-O-CO—NH, and lower alkyl-S; and
K comprises halogen, lower alkyl, lower alkyl-OH, lower perfluoroalkyl, lower alkoxy, NO₂, CN, OH, CO—OH, amino, lower alkylamine, C2-12 dialkylamine, lower acyl-O—CO—NH, lower alkoxy-CO—, and lower alkyl-S.

2. The method of claim 1, wherein the lysosomal modulating compound comprises benzyloxycarbonyl-Phe-Ala-diazomethylketone, benzyloxycarbonyl-Phe-Phe-diazomethylketone, benzyloxycarbonyl-Phe-Lys-2,4,6-trimethylbenzoyloxymethylketone, benzyloxycarbonyl-Lys-diazomethylketone, H-Gly-Phe-Gly-aldehyde semicarbazone, diazoacetyl-DL-2-aminohexanoic acid-methyl ester, a physiologically acceptable salt thereof, or a combination thereof.

3. The method of claim 1, wherein the lysosomal modulating compound comprises benzyloxycarbonyl-Phe-Ala-diazomethylketone.

4. The method of claim 1, wherein the lysosomal modulating compound is a selective antagonist for at least one cathepsin enzyme.

5. The method of claim 1, wherein n comprises an integer from 1 to 4.

6. A method of modulating lysosomal function in a subject, comprising:
administering to the subject a therapeutically effective amount of a lysosomal modulating compound selected from peptidyl diazomethylketones, peptidyl semicarbazones, diazoacetyl peptidyl alkyl esters, and physiologically acceptable salts thereof.

7. A method of reducing the risk of neurodegeneration in a subject, comprising:
administering to the subject a therapeutically effective amount of a lysosomal modulating compound, a physiologically acceptable salt of the lysosomal modulating compound, or a combination thereof, wherein enzymatic capacity of lysosomes in the subject is enhanced.

8. The method of claim 7, wherein the lysosomal modulating compound comprises M-aaₙ—CH═N═N; M-aaₙ—CH₂—O—CO—[2-R-4-R-6-R-Phenyl] (wherein each R is independently selected); M-aaₙ—NH—CH₂—CH═N—NH—CO—NH₂; or M-N ═N—CO—CH₂-aaₙ—O—R, wherein;
M comprises H, benzyloxycarbonyl (“Z”), succinyl, methyloxysuccinyl, and butyloxycarbonyl;
aa comprises a blocked or unblocked amino acid with the L configuration, D configuration, or no chirality at the alpha-carbon, the amino acid selected from alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutyric acid, epsilon-aminocaproic acid, citrulline, hydroxyproline, homoarginine, ornithine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂—CHEt₂)—COOH, alpha-aminoheptanoic acid, NH₂—CH(CH₂—1-napthyl)—COOH, NH₂—CH(CH₂—2-napthyl)—COOH, NH₂—CH(CH₂-cyclohexyl)—COOH, NH₂—CH(CH₂-cyclopentyl)—COOH, NH₂—CH(CH₂-cyclobutyl)—COOH, NH₂—CH(CH₂-cyclopropyl)—COOH, trifluoroleucine, hexafluoroleucine, phenylalanine with its phenyl mono-, di-, or trisubstituted with K, alanine with its methyl side chain replaced with a lower alkyl side chain, alanine with its methyl side chain replaced with a lower alkyl group with an attached phenyl group, alanine with its methyl side chain replaced with a lower alkyl group with two attached phenyl groups, alanine with its methyl side chain replaced with a lower alkyl group with an attached phenyl group substituted with K, and alanine with its methyl side chain replaced with a lower alkyl group with two attached phenyl groups and at least one phenyl group substituted with K;
n comprises an integer from 1 to about 20;
R comprises H, a lower alkyl group, a lower fluoroalkyl group, benzyl, a lower alkyl group substituted with J, a lower fluoroalkyl group substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, a lower alkyl group with an attached phenyl group, a lower alkyl group with two attached phenyl groups, a lower alkyl group with an attached phenyl group substituted with K, or a lower alkyl group with two attached phenyl groups and at least one phenyl group substituted with K;
J comprises halogen, COOH, OH, CN, NO₂, NH₂, lower alkyl—OH, lower alkoxy, lower alkylamine, di-lower alkylamine, lower alkoxy-CO—, lower alkyl—O—CO—NH, and lower alkyl-S—; and
K comprises halogen, lower alkyl, lower alkyl-OH, lower perfluoroalkyl, lower alkoxy, NO₂, CN, OH, CO—OH, amino, lower alkylamine, C2-12 dialkylamine, lower acyl-O—CO—NH, lower alkoxy-CO—, and lower alkyl-S.

9. A method for treating neurodegeneration in a subject, comprising:
administering to the subject a therapeutically effective amount of a lysosomal modulating compound, a physiologically acceptable salt of the lysosomal modulating compound or a combination thereof, wherein enzymatic capacity of lysosomes in the subject is enhanced.

10. The method of claim 9, wherein the enhanced enzymatic capacity is sufficient to suppress neuropathogenesis.

11. The method of claim 9, wherein the lysosomal modulating compound comprises M-aaₙ—CH═N═N; M-aaₙ—CH₂—O—CO—[2-R-4-R-6-R-Phenyl] (wherein each R is independently selected); M-aaₙ,—NH—CH₂—CH═N—NH—CO—NH₂; M-N═N—CO—CH₂- aaₙ—O—R; wherein;
M comprises H, benzyloxycarbonyl (“Z”), succinyl, methyloxysuccinyl, and butyloxycarbonyl;
aa comprises a blocked or unblocked amino acid with the L configuration, D configuration, or no chirality at the alpha-carbon, the amino acid selected from alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutyric acid, epsilon-aminocaproic acid, citrulline, hydroxyproline, homoarginine, ornithine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂—CHEt₂)—COOH, alpha-aminoheptanoic acid, NH₂—CH(CH₂—1-napthyl)—COOH, NH₂—CH(CH₂—2-napthyl)—COOH, NH₂—CH(CH₂-cyclohexyl)—COOH, NH₂—CH(CH₂—cyclopentyl)—COOH, NH₂—CH(CH₂-cyclobutyl)—COOH, NH₂—CH(CH₂—cyclopropyl)—COOH, trifluoroleucine, hexafluoroleucine, phenylalanine with its phenyl mono-, di-, or trisubstituted with K, alanine with its methyl side chain replaced with a lower alkyl side chain, alanine with its methyl side chain replaced with a lower alkyl group with an attached phenyl group, alanine with its methyl side chain replaced with a lower alkyl group with two attached phenyl groups, alanine with its methyl side chain replaced with a lower alkyl group with an attached phenyl group substituted with K, and alanine with its methyl side chain replaced with a lower alkyl group with two attached phenyl groups and at least one phenyl group substituted with K;
n comprises an integer from 1 to about 20;
R comprises H, a lower alkyl group, a lower fluoroalkyl group, benzyl, a lower alkyl group substituted with J, a lower fluoroalkyl group substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, a lower alkyl group with an attached phenyl group, a lower alkyl group with two attached phenyl groups, a lower alkyl group with an attached phenyl group substituted with K, or a lower alkyl group with two attached phenyl groups and at least one phenyl group substituted with K;
J comprises halogen, COOH, OH, CN, NO₂, NH₂, lower alkyl-OH, lower alkoxy, lower alkylamine, di-lower alkylamine, lower alkoxy-CO—, lower alkyl-O—CO—NH, and lower alkyl-S; and
K comprises halogen, lower alkyl, lower alkyl-OH, lower perfluoroalkyl, lower alkoxy, NO₂, CN, OH, CO—OH, amino, lower alkylamine, C2-12 dialkylamine, lower acyl-O—CO—NH, lower alkoxy-CO—, and lower alkyl-S.

12. The method of claim 9, wherein the lysosome modulating compound comprises at least one of benzyloxycarbonyl-Phe-Ala-diazomethylketone, benzyloxycarbonyl-Phe-Phe-diazomethylketone, benzyloxycarbonyl-Phe-Lys-2,4,6-trimethylbenzoyloxymethylketone, benzyloxycarbonyl-Lys- diazomethylketone, H-Gly-Phe-Gly-aldehyde semicarbazone, diazoacetyl-DL-2-aminohexanoic acid-methyl ester or physiologically acceptable salts thereof.

13. The method of claim 9, wherein the compound comprises benzyloxycarbonyl-Phe-Ala-diazomethylketone or physiologically acceptable salts thereof.

14. The method of claim 9, wherein the compound is a selective antagonist for at least one cathepsin enzyme.

15. A pharmaceutical preparation, including:
at least one lysosomal modulating compound, a physiologically acceptable salt of the lysosomal modulating compound, or a combination thereof; wherein the lysosomal modulating compound comprises M-aaₙ—CH═N═N; M-aaₙ—CH₂—O—CO—[2-R-4-R-6-R-Phenyl] (wherein each R is independently selected); M-aaₙ—NH—CH₂—CH═N—NH—CO—NH₂; M-N═N—CO—CH₂—aaₙ—O—R, wherein;
M comprises H, benzyloxycarbonyl (“Z”), succinyl, methyloxysuccinyl, and butyloxycarbonyl;
aa comprises a blocked or unblocked amino acid with the L configuration, D configuration, or no chirality at the alpha-carbon, the amino acid selected from alanine, valine, leucine, isoleucine, proline, methionine, methionine sulfoxide, phenylalanine, tryptophan, glycine, serine, threonine, cysteine, tyrosine, asparagine, glutamine, aspartic acid, glutamic acid, lysine, arginine, histidine, phenylglycine, beta-alanine, norleucine, norvaline, alpha-aminobutyric acid, epsilon-aminocaproic acid, citrulline, hydroxyproline, homoarginine, ornithine, sarcosine, indoline 2-carboxylic acid, 2-azetidinecarboxylic acid, pipecolinic acid (2-piperidine carboxylic acid), O-methylserine, O-ethylserine, S-methylcysteine, S-ethylcysteine, S-benzylcysteine, NH₂—CH(CH₂—CHEt₂)—COOH, alpha-aminoheptanoic acid, NH₂—CH(CH₂—1-napthyl)—COOH, NH₂—CH(CH₂—2-napthyl)—COOH, NH₂—CH(CH₂-cyclohexyl)—COOH, NH₂—CH(CH₂-cyclopentyl)—COOH, NH₂—CH(CH₂-cyclobutyl)—COOH, NH₂—CH(CH₂- cyclopropyl)—COOH, trifluoroleucine, hexafluoroleucine, phenylalanine with its phenyl mono-, di-, or trisubstituted with K, alanine with its methyl side chain replaced with a lower alkyl side chain, alanine with its methyl side chain replaced with a lower alkyl group with an attached phenyl group, alanine with its methyl side chain replaced with a lower alkyl group with two attached phenyl groups, alanine with its methyl side chain replaced with a lower alkyl group with an attached phenyl group substituted with K, and alanine with its methyl side chain replaced with a lower alkyl group with two attached phenyl groups and at least one phenyl group substituted with K;
n comprises an integer from 1 to about 20;
R comprises H, a lower alkyl group, a lower fluoroalkyl group, benzyl, a lower alkyl group substituted with J, a lower fluoroalkyl group substituted with J, 1-admantyl, 9-fluorenyl, phenyl, phenyl substituted with K, phenyl disubstituted with K, phenyl trisubstituted with K, naphthyl, naphthyl substituted with K, naphthyl disubstituted with K, naphthyl trisubstituted with K, a lower alkyl group with an attached phenyl group, a lower alkyl group with two attached phenyl groups, a lower alkyl group with an attached phenyl group substituted with K, or a lower alkyl group with two attached phenyl groups and at least one phenyl group substituted with K;
J comprises halogen, COOH, OH, CN, NO₂, NH₂, lower alkyl-OH, lower alkoxy, lower alkylamine, di-lower alkylamine, lower alkoxy-CO—, lower alkyl-O—CO—NH, and lower alkyl-S—; and
K comprises halogen, lower alkyl, lower alkyl—OH, lower perfluoroalkyl, lower alkoxy, NO₂, CN, OH, CO—OH, amino, lower alkylamine, C2-12 dialkylamine, lower acyl-O—CO—NH, lower alkoxy-CO—, and lower alkyl-S—.

16. The pharmaceutical preparation of claim 15, wherein the compound is administered to a subject at a therapeutically effective amount to modulate cellular content of lysosomes.

17. The pharmaceutical preparation of claim 15, wherein the compound comprises at least one of benzyloxycarbonyl-Phe-Ala-diazomethylketone, benzyloxycarbonyl-Phe-Phe-diazomethylketone, benzyloxycarbonyl-Phe-Lys-2,4,6-trimethylbenzoyloxymethylketone, benzyloxycarbonyl-Lys- diazomethylketone, H-Gly-Phe-Gly-aldehyde semicarbazone, diazoacetyl-DL-2-aminohexanoic acid-methyl ester, or physiologically acceptable salts thereof.

18. The pharmaceutical preparation of claim 15, wherein the compound comprises benzyloxycarbonyl-Phe-Ala-diazomethylketone or physiologically acceptable salts thereof.

19. The pharmaceutical preparation of claim 15, wherein the compound is a selective antagonist for at least one cathepsin enzyme.

20. A method of studying lysosomal function in vitro, comprising:
providing a tissue culture;
inducing lysosomal dysfunction with an appropriate lysosomal disrupter;
applying a lysosomal modulating compound to the culture; and
monitoring transport markers and synaptic recovery.
